(19) United States
(12) Patent Application Publication
(10) Pub. No.: US 2009/0137980 A1
(43) Pub. Date: May 28, 2009

(54) METHOD AND APPARATUS FOR MULTI-INPUT STEPWISE INFUSION PRESCRIPTION

(75) Inventor: Irfan Z. Ali, Woodbury, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/324,434

(22) Filed: Nov. 26, 2008

Related U.S. Application Data
(60) Provisional application No. 60/990,669, filed on Nov. 28, 2007.

(51) Int. Cl. A61M 5/168

(52) U.S. Cl. 604/503; 604/66

(57) ABSTRACT

A drug infusion system includes a drug delivery module which delivers a drug in each of a series of sequential time slots over a period of time. The drug delivery module is controlled by a controller which is configured to cause the drug delivery module to deliver said drug in the series of sequential time slots in a manner defined by a formula in which a quantitative characteristic of drug delivery in each time slot is a function of a past drug delivery profile and at least one of: a) a medical professional-provided profile, b) a patient-chosen parameter, and c) a non-variable parameter. The formula may be a function of a medical professional-provided profile and a non-variable parameter may be a parameter chosen by a medical professional. The past drug delivery profile may include a drug delivery parameter in a previous time slot. The patient-chosen parameter may include an input from the patient characterizing the patient's condition.

### FIELD OF THE INVENTION

This invention relates to drug infusion devices and, in particular, implantable drug infusion devices that are programmable by a medical professional.

### TECHNICAL BACKGROUND

Drug infusion devices dispense fluid medication, containing a drug, to a patient. Some drug infusion devices are portable, allowing a patient to receive fluid medication while remaining mobile. In addition, some drug infusion devices are implanted in the patient's body to more effectively and less obtrusively dispense such fluid medication to a patient.

Various devices and techniques for treating a patient by drug infusion are disclosed in, for example, U.S. Pat. No. 5,782,798, entitled Techniques For Treating Eating Disorders By Brain Stimulation and Drug Infusion; U.S. Pat. No. 5,814,014, entitled Techniques of Treating Neurodegnerative Disorders by Brain Infusion; and U.S. Pat. Nos. 6,579,280 and 7,008,413, each entitled Generic Multi-Step Therapeutic Treatment Protocol. All of these are assigned to Medtronic, Inc. of Minneapolis, Minn., and all are hereby incorporated by reference.

U.S. Pat. No. 4,146,029, Ellinwood, Self-Powered Implanted Programmable Medication System and Method, discloses a device and method for dispensing medication internally of the body utilizing an implanted system which includes medication storage and dispensing control circuitry having control components which may be modified by means external of the body being treated to control the manner of dispensing the medication within such body. In particular, extracorporeal control means may provide some measure to achieve selected medication programs corresponding to particular codes.

An implantable drug administration device may be non-invasively programmed to change the dosage amount and/or the dosage delivery rate. A medical professional may program the delivery rate of a drug contained in the reservoir of the device over a specified interval.

Implantable drug infusion devices and systems are commonly programmable with a plurality of programming steps. Each programming step typically is conducted for a specific time or a specific period of time and specifies an amount of fluid medication or a rate of delivery of fluid medication to a patient. A plurality of programming steps can typically be sequenced to create a programming cycle delivering fluid medication to a patient at different rates based on a daily, weekly, or other time-based schedule.

A program cycle is typically designed, i.e., planned and developed, to cover a set of known time periods, e.g., a period of one week. Each day of the week could be separately programmed or could be based on repetition of a daily program. For example, one could program a program to be repeated each week day and a different program to run on each day of the weekend, for example. Each step in the program cycle, perhaps each hour of the day, could have a different programmed delivery amount or delivery rate. As an example, drug infusion devices could deliver more pain medication during daytime hours when a patient is more active. Other patient activity schedules can and are accommodated, such as non-daily, weekly schedules.

Upon implantation of the drug infusion device, the device may need to be programmed, i.e., a new or modified programming cycle may need to be installed, loaded and/or activated. Or, not infrequently, the drug infusion device may need to be adjusted or readjusted to take into account variations in the patient's condition and/or the patient's activities, for example. These situations typically involve programming a cycle which takes effect immediately or at some discrete point in time in the future.

### SUMMARY OF THE INVENTION

In one embodiment, a drug infusion system including a drug infusion device capable of delivering a drug to a patient includes a drug delivery module capable of delivering the drug to the patient at one of a series of dosages in each of a series of sequential time slots over a period of time, and a controller operatively coupled to the drug delivery module to control delivery rates at which the drug is delivered to the patient. The controller is configured to cause the drug delivery module to deliver the drug in the series of sequential time slots in a manner defined by a formula in which at least one quantitative drug delivery characteristic is a function of a past drug delivery profile and at least one of: a) a medical professional-provided profile, b) a patient-chosen parameter, and c) a non-variable parameter. The at least one quantitative drug delivery characteristic may include at least one of: a) an amount of drug delivered at each time slot, b) a delivery rate, and c) duration of drug delivery, in each time slot.

The formula may be a function of a medical professional-provided profile and the non-variable parameter, (for example, a constant quantity or constant percentage increase) is a parameter chosen by the medical professional. The formula may include a seed value, i.e., a starting point for drug delivery rate, supplied by the medical professional. The seed value may be, for example, a definite numerical value for delivery rate or dosage, or it could be a function of a past drug delivery interval (for example, a defined percentage of drug delivery rate at a particular point in the past or an average delivery rate over a defined previous time period), or a previous flex prescription. The medical professional-provided profile may include conditions governing a delivery parameter such as (for example) an upper or a lower limit for delivery rate, or a range for delivery rate.

The past drug delivery profile may include a drug delivery parameter in a previous time slot.

The patient-chosen parameter may include an input from the patient characterizing the patient's condition. The patient input may be a rating of pain experienced by the patient or a rating of involuntary movement being experienced by the patient. The input may be an indication of the patient's desire for a change in quantity of drug (for example, a desire for an administration of a bolus).

The controller may be further configured to cause the delivery module to deliver a bolus in response to the user input, wherein the controller interrupts the series of sequential time slots and deliver the bolus during the interruption. Alternatively, the controller may be configured to cause the module to deliver a bolus wherein the drug delivered in the bolus is in addition to the drug delivered according to the formula during the duration of the bolus. The controller may be further configured to cause the delivery device to resume the series of sequential time slots after the bolus delivery, starting at the time slot corresponding to the elapsed time since the initial start of the series.

In another aspect, the non-variable parameter of the function may include a percent rate of change of at least one quantitative characteristic of drug delivery from a previous time slot.

The duration of each time slot in the series of time slots may be fixed, or alternatively, may be variable. The number of time slots in the series of sequential time slots may be fixed or may be variable.

In another aspect, the controller includes a memory unit capable of storing information relating to the formula; and a programmer which is external to the patient and which includes a user interface configured to receive an input from a user; the memory unit and the programmer being adapted for communication. The communication may be RF or inductive communication, or any other communication mode.

In another aspect, a method of delivering a drug to a patient by an implantable drug infusion device includes: delivering the drug to the patient at one of a series of dosages in each of a series of sequential time slots over a period of time; controlling the delivery of the drug in a manner defined by a formula in which at least one of a) an amount of drug delivered at each time slot, b) a delivery rate, and c) duration of drug delivery, is a function of a past drug delivery profile and at least one of: a) a medical professional-provided profile, b) a patient-chosen parameter, and c) a non-variable parameter. They may also be functions of a specified seed value, or starting value, as well.

In the method, the formula may be a function of a medical professional-provided profile and a non-variable parameter is a parameter chosen by a medical professional. The patient-chosen parameter may include an input from the patient characterizing the patient's condition. The patient input may be a rating of at least one of pain experienced by the patient; involuntary movement; and desire for a drug bolus.

The non-variable parameter of the function may include a percent rate of change of at least one of a quantitative characteristic of drug delivery from a previous time slot. In addition, the non-variable parameter of the function may include an increment of change of at least one of a quantitative characteristic of drug delivery in a previous time slot. The time slots may be of unequal duration.

The device and methods described herein make it possible to provide a drug dosage regimen which includes non-uniform incremental changes in delivered dosage. This is desirable in cases where an abrupt transition in dosage or an abrupt start-up or cessation of drug administration results in undesirable effects on the patient.

Additional features and advantages of the invention will be set forth in the detailed description which follows, and in part will be readily apparent to those skilled in the art from the description or recognized by practicing the invention as described in the written description and claims hereof, as well as in the appended drawings. It is to be understood that both the foregoing general description and the following detailed description are merely exemplary of the invention, and are intended to provide an overview or framework for understanding the nature and character of the invention as it is claimed. The accompanying drawings are included to provide a further understanding of the invention, and are incorporated in and constitute a part of this specification. The drawings are not necessarily to scale, and sizes of various elements may be distorted for clarity. The drawings illustrate one or more embodiment(s) of the invention, and together with the description serve to explain the principles and operation of the invention.

### BRIEF DESCRIPTION OF THE DRAWINGS

FIG. 1 is a schematic view of a patient with a drug infusion device implanted within the patient's body and its associated external programmer.

FIG. 2 is a schematic representation of the clinical programmer of FIG. 1.

FIG. 3 is a schematic representation of the patient programmer of FIG. 1.

FIG. 4 is a block diagram of the drug infusion system of FIG. 1.

FIG. 5 is a plot of fluid delivery rate vs. time, illustrating a drug delivery protocol with a step-wise increasing delivery rate.

FIG. 6 is a plot of fluid delivery rate vs. time illustrating a drug delivery protocol with a step-wise decreasing delivery rate.

FIG. 7 is a plot of fluid delivery rate vs. time illustrating a drug delivery protocol with a step-wise increasing delivery rate and a bolus.

FIG. 8 is a plot of fluid delivery rate vs. time illustrating a non-uniform incrementally stepped drug delivery protocol which transitions to a flex drug prescription.

FIG. 9 is a plot of fluid delivery rate vs. time illustrating a drug delivery protocol having varying time slot duration.

FIG. 10 is a plot of fluid delivery rate vs. time illustrating a drug delivery protocol with repeated step groups.

FIG. 11 is a flow chart illustrating a method of controlling drug infusion.

FIG. 12 is a plot of delivery rate vs. time illustrating a drug delivery protocol which is a function of physiologic sensor data.

### DETAILED DESCRIPTION OF THE INVENTION

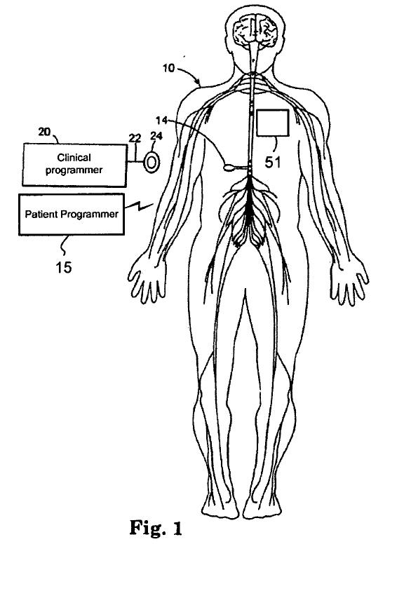

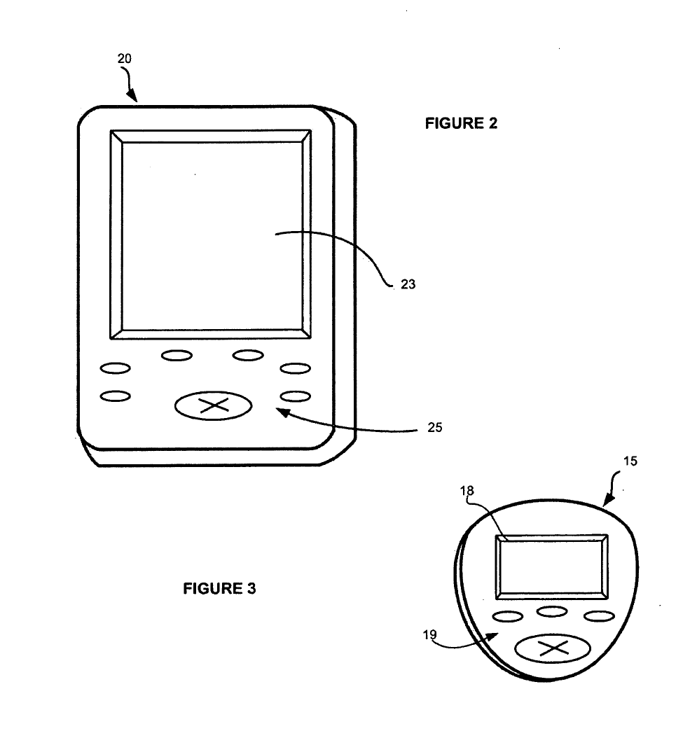

FIG. 1 is a schematic view of a drug infusion system. In the illustrated embodiment, a drug infusion device 14 is implanted within the body of a patient 10. The illustrated drug infusion device 14 is programmable through a telemetry link from an external clinical programmer 20, which is coupled via a conductor 22 to a radio frequency antenna 24. Although an RF antenna is illustrated, it will be understood that other communications technologies such as inductive communication, or other communication modes, may be used. A patient programmer 15 may also be provided to communicate patient input to the implanted infusion device 14 via RF, inductive communication, or other communication modes. Referring to FIG. 2, the illustrated clinical programmer 20 has a user interface which includes a display screen 23 and an input keypad 25, through which a user such as a medical professional inputs information on the medication infusion protocol to be programmed into the device 14. As shown in FIG. 3, the patient programmer 15 includes a screen 18 and keypad 19 which make up a user interface. The clinical programming operation and patient input are discussed in more detail below. The drug infusion device 14 may include, for example, a pump for infusing a fluid, such as a fluid medication, into the patient's body. Implantable infusion devices and programmers which communicate with the implanted infusion devices using inductive coupling telemetry in order to program the devices include, for example, the infusion devices and programmers sold under the trademarks Synchromed, Synchromed EL, and Synchromed II, the programmer sold under the trademark N′Vision by Medtronic, Inc. of Minneapolis, Minn., and the PTM (patient therapy manager) patient programmer sold by Medtronic, Inc. of Minneapolis, Minn.

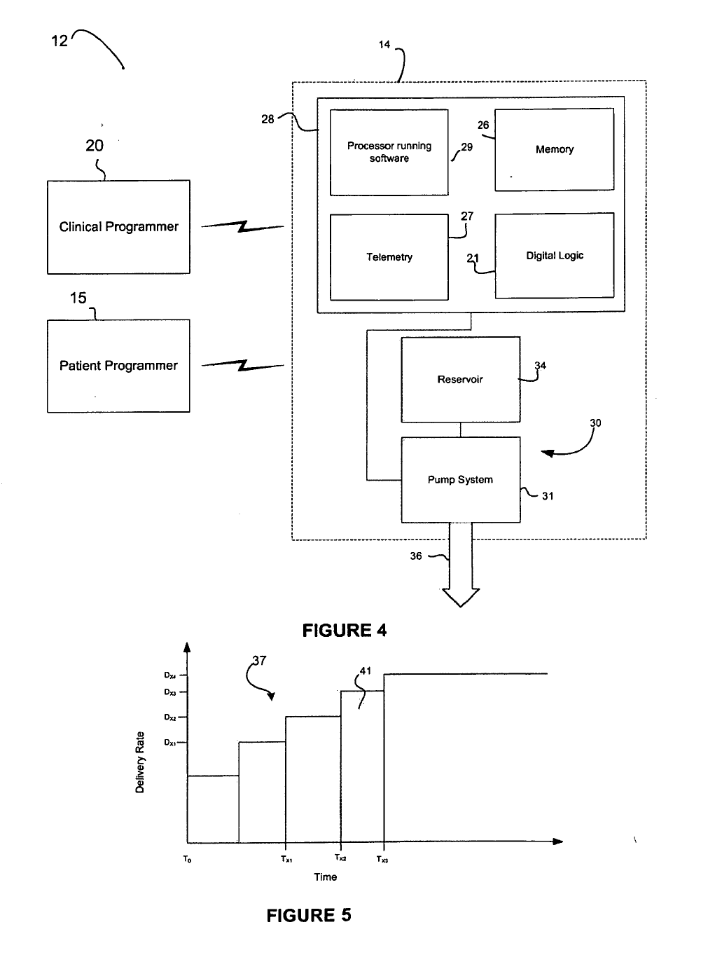

FIG. 4 is a block diagram of the drug infusion system 12 having an implantable drug infusion device 14. In the illustrated embodiment, drug infusion device 14 includes an internal controller 28 which includes a memory unit 26, a processor 29 running software, and a telemetry module 27. The internal controller 28 may also include a digital logic module 21. The processor 29, memory 26, telemetry module 27 and digital logic module 21 communicate among themselves via conventional means such as a bus. The implantable device 14 also includes a power source such as a primary cell or rechargeable battery (not shown). The telemetry module 27 receives input from the clinical programmer 20 and the patient programmer 15. These inputs are communicated to the processor 29. The processor 29 runs software with which the internal controller 28 provides drug delivery instructions to a drug delivery module 30 which includes a pump system 31. The pump system 31 includes a pump for infusing a fluid medication, including a drug or a combination of drugs, to patient 10. A reservoir 34 in fluid communication with the pump system 31 holds fluid medication to be infused to the patient through catheter tubing 36. Such drug delivery modules 30 are well known in the art. In the illustrated embodiment, the drug delivery module 30 operates according to a dosing regimen under the control of the internal controller 28 for infusing a fluid to the patient 10.

During a programming operation, the internal controller 28 receives programming information, via telemetry, from the clinical programmer 20. Programming information is stored in the memory unit 26 of the controller 28. As described in greater detail below, the processor 29 performs algorithms and other operations on the supplied information to determine the dosing regimen to be performed by drug delivery module 30. Drugs may be provided to a patient 10 by drug delivery module 30 at one or more predetermined dosages, generally calculated as a delivery rate, typically specified as an amount of drug provided to patient 10 in a defined period of time. For example, the dosage may be specified as an amount of drug (measured in, for example, milligrams) per twenty-four hour period. U.S. Pat. No. 7,008,413 entitled “Generic Multi-Step Therapeutic Treatment Protocol”, and U.S. Published Patent Application No. 2005/0043863 entitled “Drug Infusion System and Method Adapted to Start During Programming Cycle”, both of which are hereby incorporated by reference, describe methods of programming a dosing regimen.

Some drugs may have undesirable side effects when dosage is abruptly started or significantly increased that can be minimized by a gradual ramp-up of the dosage delivered. This may be accomplished by starting drug delivery at a relatively low dosage and increasing the dosage in a step-wise fashion over a period of time. With some medications, there may be patient discomfort or other undesirable effects when administration of the drug is abruptly stopped or dosage is significantly and abruptly decreased. In some cases, a gradual tapering down of dosage (rather than an abrupt halt or drop in dosage) may ease the discomfort and other undesirable effects. This tapering down may be accomplished in a step-wise fashion. Another situation where an incrementally stepped increase in dosage is one where a patient is drug resistant. A stepped increase or decrease in dosage may also be desirable for a newly implanted patient who will be transitioned to a flex prescription, for an established infusion patient transitioning to a new flex prescription, or for an infusion patient being transitioned to a different drug or a different drug concentration.

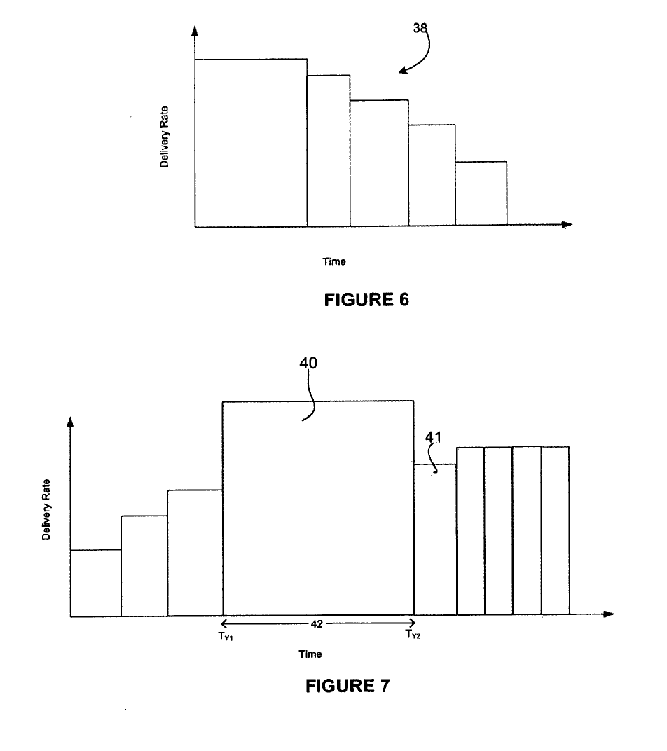

FIG. 5 shows an example of a step-wise increase in drug dosage that may be delivered with the drug infusion device 14, and FIG. 6 shows an example of a step-wise decrease in drug dosage that may be delivered with the device. In these examples, the controller 28 in the implantable infusion device 14 is configured to cause the drug delivery module 30 to deliver the fluid in a series of sequential time slots in a manner in which a quantitative delivery characteristic is varied from one time slot to the next. The varied delivery characteristic may be any one or more of delivery rate, dosage (or volume delivered), time slot duration, change in delivery rate from a previous time slot, change in dosages (or volume delivered) from a previous time slot, change in time slot duration from a previous time slot. The number of time slots in the stepped protocol may vary, and would be chosen by the medical professional. In the examples shown, the delivery rate at which fluid medication is delivered in each of the series of sequential time slots is a monotonically increasing (FIG. 5) or a monotonically decreasing (FIG. 6) function of the number of time slots that have transpired, i.e., the delivery rate in the step-up embodiment does not decrease from step to step, and in the step-down embodiment, it does not increase from step to step.

The delivery rate in successive steps need not increase (or decrease) in a uniform manner. The change in this parameter from step to step may be non-uniform. For example, in FIG. 5, (Dₓ₂−Dₓ₁) is not equal to (Dₓ₃−Dₓ₂). Other drug delivery characteristics may also be non-uniform. For example in FIG. 5, the duration of the time slots is non-uniform; (Tₓ₂−Tₓ₁) is not equal to (Tₓ₃−Tₓ₂).

In the embodiment illustrated in FIG. 5, delivery rate is stepped up until, at a point in time Tx₃, the maximum desired delivery rate Dₓ₄ is reached. After this point in time, delivery rate is held steady. Variations in delivery rate step decreases and time slot durations can be seen in the protocol illustrated in FIG. 6 as well. The drug delivery profile of the final step could extend indefinitely. For example, the drug delivery profile can provide that after some particular step, the dosage of that step be continued indefinitely. In another alternative, a group of steps could be repeated. This involves a programming in of a delivery profile such as: “after step 10, repeat steps 5-10”. FIG. 10 illustrates an example of a profile where a group of steps 50 (steps 4, 5 and 6) are repeated two more times as groups 51 (steps 4′, 5′, 6′) and 52 (steps 4″, 5″, 6″). Each of the step groups 50, 1 and 52 have been filled with a different pattern to make the group repetition clearer. The illustrated repetition can be defined as: “after step 6, repeat steps 4-6 two more times”. Although the repeated groups have been shown to be adjacent each other in the dosing sequence, they could, alternatively, be interspersed with non-repetitive steps.

The drug delivery protocol may be a function of a medical professional-provided profile. The medical professional-provided profile may include conditions governing a delivery parameter such as, for example, an upper limit for delivery rate which will not be exceeded (delivery rate<Y), or a lower limit under which delivery rate will not fall (delivery rate>X), or a range for delivery rate (X<delivery rate<Y).

The protocol illustrated in FIG. 5 may be useful where a gradual ramping up of drug dosage is desired in order to avoid negative side effects or discomfort which may be caused by a more sudden increase of dosage. The protocol illustrated in FIG. 6 may be useful where a decrease in dosage or cessation of drug delivery is desired, but where a gradual stepping down of delivered dosage is desired to minimize discomfort due to the withdrawal of medication delivery. For applications where drug dosage is being ramped up or ramped down, the desired change from step to step may be relatively small. The changes can be proportional to the last step (for example, dosage increased/decreased by X % from the previous step), or can be of a fixed quantity (for example, an increase of 5.5 mg, 6 mg, 7 mg, 9 mg over the previous drug dosage or over the dosage in a specified time slot, in each of four time slots, respectively). In another alternative, increases or decreases may be based, at least in part, on the patient's self-assessment. For example, if the patient reports via the patient programmer 15, that pain, involuntary movement, or some other condition is at a particular level (e.g., pain is a “7” on a scale of 1 to 10, or involuntary movement is at “medium” on a high-medium-low scale), the internal controller 28 may be programmed to respond by causing drug dosage increases in steps of a particular magnitude or percentage corresponding to the patient's self-assessment rating, up to a maximum set by the clinician.

While medication delivery is being ramped up or ramped down, it may be desirable to administer a bolus of medication at certain times and, in some cases, to allow the patient to self-administer a bolus. FIG. 7 illustrates the step-up protocol of FIG. 5 with a bolus 40 delivered in the time slot 42 which lies between Tx₁ and Tx₂. In the illustrated embodiment, after delivery of the bolus 40 is completed, the step-wise protocol is resumed at time Tx₂ at the point in the step sequence where it had been interrupted by the bolus 40, i.e., at the fifth step 41. Alternatively, after delivery of the bolus 40, the step-up protocol could be restarted from its initial step, or it could be resumed at the point in the stepped protocol that would have corresponded to time Tx₂ in the absence of bolus delivery. A step-down protocol, as illustrated in FIG. 6, likewise may be interrupted by bolus delivery in any of the alternative manners described above. Bolus delivery may be preprogrammed into a step-up or step-down protocol, or the patient may be provided with a user interface which communicates with the controller 28 to release one or more boluses at the patient's option. Parameters for bolus administration (for example, number of boluses permitted, bolus delivery rate, and/or bolus duration) by the patient may be determined and programmed by a medical professional. A bolus may be incremental, i.e., an addition to on-going drug delivery, or it may be exclusive, i.e., replacing the preprogrammed drug delivery for a period of time. The drug delivery program could then be resumed at the point it was interrupted by the exclusive bolus, or if desired, it could resume at the point where it would have been had the bolus not been delivered.

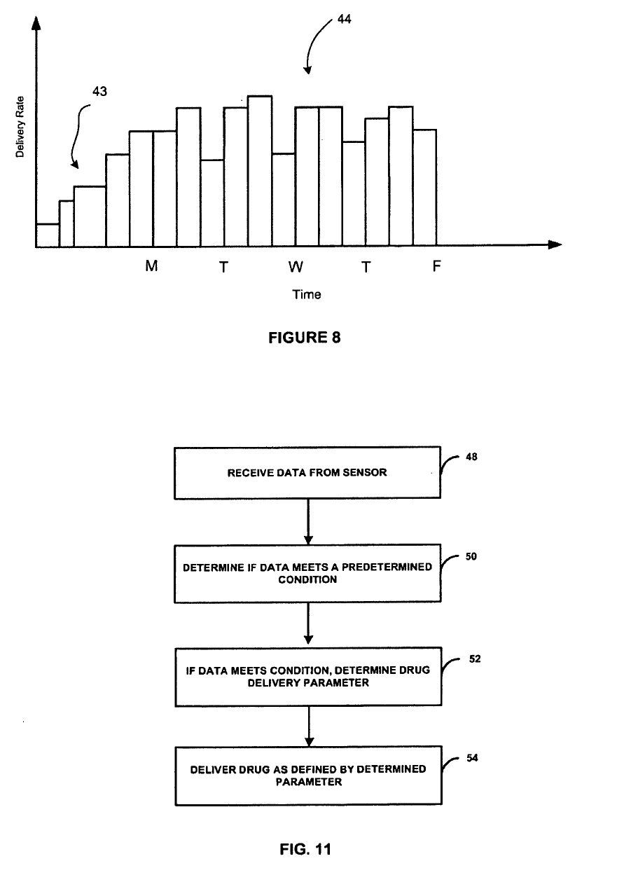

Once protocol is completed, the infusion device can be instructed to continue a particular dosage for an indefinite period of time. Or, a flex prescription may be commenced once a stepped program is completed. This is illustrated in FIG. 8, where a non-uniform step drug infusion regimen 43 is administered from time T₀ until time M, at which point a flex prescription 44 is commenced. In the illustrated flex prescription, the notations M, T, W, T, F refer to days of the week. Other alternatives at completion of the protocol include a repetition of all or a selected group of steps in the protocol (discussed above), or stopping the pump.

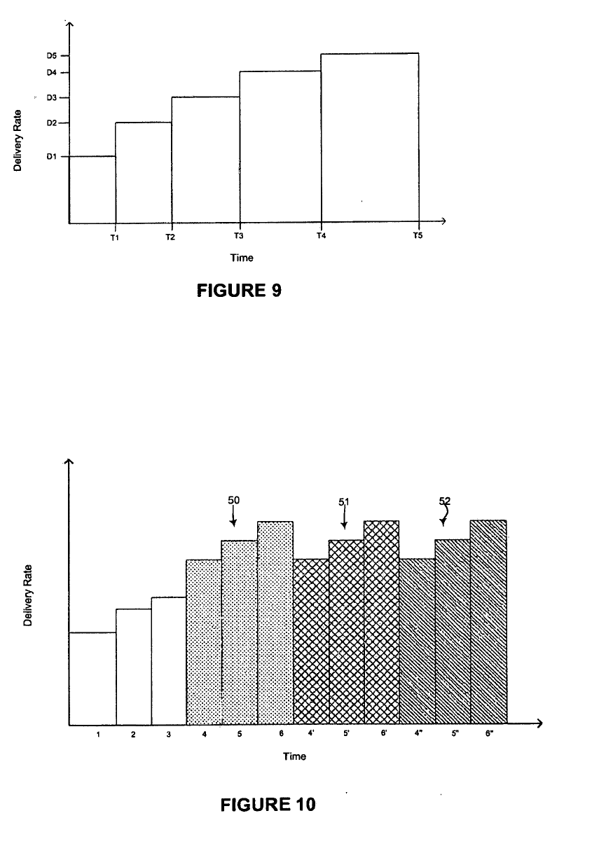

One or more drug delivery parameter (for example, amount of drug to be delivered at each time slot, or the delivery rate in each time slot, or the duration of drug delivery) may be based on an equation or formula input by the user. FIG. 9 illustrates a protocol where duration of time slots is not a constant but instead varies from one time slot to the next. In the illustrated protocol, the duration of time slots increase by 20% over the duration of the previous time slot. This is but one example of how time slot duration may be based on an equation or formula. Whichever drug delivery parameter is varied, the formula may also include a non-variable parameter. For example, a medical profession may program in a formula to provide an X % increase in duration and Y % increase in dosage for each of Z steps, where X, Y and Z are numerical constants. The formula may be input via a user interface on the programmer 20. A conversational interface for a programmer is described in detail in U.S. Published Patent Application No. 2006/0041288, which is hereby incorporated by reference in its entirety. A conversational interface may be presented on the display 23 (which is preferably a touch screen) of the programmer 20 to elicit from the medical professional the necessary information. The medical professional responds to a series of queries either through the keypad 25 or through interaction with the display touch screen 23.

The following is an illustration of a script for programming a non-uniform incrementally stepped dosage regimen:
Interface queries: “1. Do you want to program an incremental protocol? Yes/No”
User chooses “Yes”.
Interface queries: “2. Starting dose: ______.”
User inputs a numerical value.
Interface queries: “3. Change drug delivered in each time slot/delivery rate/duration of time slot [alternatives given in a dropdown menu] by ______% [alternatives given in a dropdown menu] for each of the next ______ time slots/until a total rate of ______ is reached/until a total volume of ______ is delivered.”
User chooses the desired parameter(s) via the dropdown menu. User inputs numerical values in the blanks. User inputs a positive value for an increase and a negative value for a decrease.
Interface queries: “4. Do you want to add additional time slots programmed with incremental/constant/flex [alternatives given in a drop-down menu] protocol? Yes/no.”
User chooses “yes” and chooses “incremental” from drop down menu
Interface repeats query 3 and 4. (Had “constant” been chosen, interface could present, for example, the following query: “6: Maintain dosage of last time slot for a period of ______.” Had “flex” been chosen, the interface would begin a script for programming in a flex prescription.)
User inputs a definite time or an input signifying “indefinite”.

The above script is a particular programming scenario presented for purposes of illustration. Those skilled in the art will appreciate that alternative programming scenarios may be programmed by providing appropriate queries to elicit the information needed from the medical professional.

In another alternative, a dosage regimen is provided based on an equation or formula programmed by the medical professional and also on one or more patient-entered input(s) indicating the patient's assessment of their condition. For example, the patient may enter a pain score or a self-assessment of severity of involuntary movement, or may input a desire for a bolus on the patient programmer 15 illustrated in FIG. 3. The user interface screen 18 of the patient programmer 15 may present, for example, a numerical scale for self-assessment of pain, involuntary movement or other patient condition. Alternatively or in addition, it may present a graphical analog scale (for example, face icons or line drawings showing representations corresponding to levels of discomfort) on which the patient may indicate his perception of his condition. The patient inputs his self-assessment via the keypad 19 or through interaction with screen 18 (where screen 18 is a touch screen). Drug infusion device response to a patient input may be based on parameters input to the infusion device by the medical professional. These parameters may include, for example, the parameters governing administration of a bolus when a patient self-assessment input is at or above a chosen threshold.

The processor 29 in controller 28 is configured to determine a dosing parameter by applying an algorithm to information input by the medical professional. For example, if the medical professional inputs a desired initial delivery rate, a desired final delivery rate (i.e., the delivery rate to be reached at the end of the stepped protocol) and the time frame desired to transition from the initial rate to the final rate, the processor 29 can run an algorithm to determine, for example, the number of steps, the duration of each step, and the increase (or decrease) in delivery rate for each step. The algorithm may be designed so that one or more parameters are held constant while others may be varied. For example, the number of time slots or duration of individual time slots may be fixed in one embodiment, or may alternatively be subject to the user's choice. The inputs may also or alternatively include how many steps are desired; the overall change in dosage expressed as a percentage of a dosage used as a starting point or seed value; and/or the total volume to be delivered. These factors and other like them will be used by the algorithm to determine the end point for the drug delivery program to be configured by the algorithm. Alternatively, a processor in the clinical programmer or a combination of a processor in the clinical programmer and the processor 29 in the implantable device can determine the dosing parameter by applying one or more algorithms to information input by the medical professional.

Referring to FIG. 1, a sensor 51 that senses a physiological parameter indicative of the patient's condition may be provided. Preferably, the sensor would sense a patient physiological parameter that is indicative of the patient's need for or reaction to the medication delivered by the patient's drug infusion device. The sensor may be external to the patient's body or may be implanted within the patient. Examples of such sensors include, but are not limited to, a set of electrodes to sense electrical activity in the patient's heart or brain, a blood glucose sensor, a respiration rate sensor, a temperature sensor, or a motion detector (e.g., an accelerometer) to detect patient movement such as a tremor, change in posture or activity level.

In one embodiment, sensor 51 is an accelerometer, for example, a 3-axis accelerometer which can provide position data and data on rate of movement. The sensor transmits data to the controller 28 in the infusion device 14. The processor 29 in the controller 28 analyzes the sensor data for use in determining the dosage regimen to be administered, or the need for administration of a bolus. Alternatively, sensor data may also be transmitted to the patient programmer 15. Referring to FIG. 11, in an embodiment where the physiological sensor data is used to determine dosing regimen, the drug infusion device 14 receives the sensor data (48) and determines if the data meets a predefined condition (50). This predefined condition may be, for example, a quantity in the data above a threshold. If the data meets the condition, then the infusion protocol parameter(s) corresponding to that condition being met are determined (52). The drug is then delivered according to the determined protocol (54). A protocol with delivery rates, number of time slots in a step series, or duration of each time slot in the series, based at least in part on the information received from the sensor, will be delivered. The determination may be made, for example, by means of a look-up table stored in the memory unit 26 which stores a particular infusion protocol corresponding to a threshold value being reached by the sensor data.

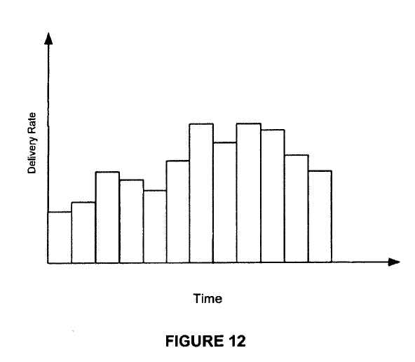

In one embodiment, the sensor is configured to sense physiological parameters associated with tremor and levels of tremor. The sensor data is transmitted to the controller and if sensor data indicates that a particular level of tremor is present, then a bolus having pre-defined characteristics may be delivered, instead or in addition to the drug being delivered according to the scheduled protocol. As another alternative, a stepped-up drug infusion regimen such as that illustrated in FIG. 5 may be initiated upon sensor data meeting a predefined condition which is indicative of a patient's need for the stepped-up protocol. For example, a significant increase or decrease in activity may indicate a patient is experiencing a change in pain level and indicate the patient's need for the stepped-up protocol. These alternatives are for illustrative purposes only; other responses to a patient's condition as indicated by sensor data, in the form of a change to the ongoing drug delivery regimen, may be programmed into the drug infusion system. FIG. 12 illustrates an example of a drug delivery profile where delivery varies based at least in part on sensor data. Because sensor data input is based on patient condition at various points in time and not on a formula or rule, sensor data input may lend a somewhat irregular appearance to the delivery profile.

It will be recognized and understood that the dosing protocols illustrated and described above are examples only and many other dosing regimens incorporating non-uniform incremental changes are possible.

While the embodiments of the present invention have been described in terms of an implantable drug infusion device, it is to be recognized and understood that the features of the present invention could also be implemented in a non-implantable drug infusion device. An example of a non-implantable programmable drug infusion device is that sold under the trademark Paradigm by Medtronic, Inc.

One skilled in the art will appreciate that the present invention can be practiced with embodiments other than those disclosed. The disclosed embodiments are presented for purposes of illustration and not limitation. Various modifications and variations can be made to the present invention without departing from the spirit and scope of the invention. Thus, it is intended that the present invention cover the modifications and variations of this invention provided they come within the scope of the appended claims and their equivalents.

What is claimed is:

1. A drug infusion system for delivering a drug to a patient, comprising:
a drug delivery module configured to deliver said drug to said patient at one of a series of dosages in each of a series of sequential time slots over a period of time;
a sensor which senses a patient physiological parameter; and
a controller in communication with the sensor and operatively coupled to said drug delivery module to control delivery rates at which said drug is delivered to said patient; wherein said controller is configured to cause the drug delivery module to deliver said drug in the series of sequential time slots in a manner defined by a formula in which a quantitative drug delivery parameter for each time slot is a function of a seed value, the sensed patent parameter and at least one of:
a) a medical professional-provided profile,
b) a patient-chosen parameter, and
c) a non-variable parameter.

2. The system of claim 1 wherein the sensor includes at least one of: a set of electrodes to sense electrical activity in the patient's heart; a set of electrodes to sense electrical activity in the patient's brain, a blood glucose sensor, a temperature sensor, a respiration rate sensor, or a motion detector.

3. The system of claim 1 wherein the formula is a function of a medical professional-provided profile and a non-variable parameter is a parameter chosen by a medical professional.

4. The system of claim 1 wherein the patient-chosen parameter includes an input from the patient characterizing the patient's condition.

5. The system of claim 4 wherein the patient input is a rating of pain experienced by the patient.

6. The system of claim 5 wherein the rating of pain is input with reference to a graphical pain scale representation.

7. The system of claim 4 wherein the patient input is a rating of involuntary movement being experienced by the patient.

8. The system of claim 4 wherein the patient input is an indication of the patient's desire for a change in quantity of drug.

9. The system of claim 1 wherein the non-variable parameter of the function includes a percent rate of change of at least one of a quantitative parameter of drug delivery in a previous time slot.

10. The system of claim 1 wherein the controller is further configured to cause the delivery module to deliver a bolus in response to the sensed patient physiological parameter data meeting a predefined condition.

11. The system of claim 10 wherein the controller is further configured to cause the module to deliver a bolus wherein the drug delivered in the bolus is in addition to the drug delivered according to the formula during the duration of the bolus.

12. The system of claim 10 wherein the controller is further configured to cause the delivery device to resume the series of sequential time slots after the bolus delivery, starting at the time slot corresponding to the elapsed time since the initial start of the series.

13. The system of claim 1 wherein the duration of each time slot in the series of time slots is fixed.

14. The system of claim 1 wherein the duration of each time slot in the series of time slots is variable.

15. The system of claim 1 wherein the number of time slots in the series of sequential time slots is fixed.

16. The system of claim 1 wherein the number of time slots in the series of sequential time slots is variable.

17. The system of claim 1 wherein the controller includes
a memory unit capable of storing information relating to the formula; and
a processor capable of storing instructions for configuring a drug delivery profile; and
a telemetry module for communication with an external programmer.

18. The system of claim 1 wherein the sensor is located within the body of the patient and separated from the drug delivery module.

19. The system of claim 1, wherein at least two time slots in the series of sequential time slots differ in duration.

20. A drug infusion device capable of delivering a drug to a patient, comprising:
a drug delivery module capable of delivering said drug to said patient at one of a series of dosages in each of a series of sequential time slots over a period of time;
a sensor which senses a patient physiological parameter; and
a controller in communication with the sensor and operatively coupled to said drug delivery module to control delivery rates at which said drug is delivered to said patient; wherein said controller is configured to cause the drug delivery module to deliver said drug in the series of sequential time slots in a manner defined by a formula in which a quantitative drug delivery parameter for each time slot is a function of a seed value, the sensed patent parameter and at least one of:
a) a medical professional-provided profile,
b) a patient-chosen parameter, and
c) a non-variable parameter.

21. A method of delivering a drug to a patient by a drug infusion system comprising:
sensing a patient physiological parameter with a sensor;
delivering said drug to said patient with an implanted drug delivery module at one of a series of dosages in each of a series of sequential time slots over a period of time; and
controlling delivery rates at which said drug is delivered to said patient with a controller that is in communication with the sensor and operatively coupled to said drug delivery module to cause the drug delivery module to deliver the drug in a-manner defined by a formula in which a quantitative characteristic of drug delivery is a function of the sensed patient physiological parameter and a seed value and at least one of:
a) a medical professional-provided profile,
b) a patient-chosen parameter, and
c) a non-variable parameter.

22. The method of claim 21 wherein the formula is a function of a medical professional-provided profile and a non-variable parameter is a parameter chosen by a medical professional.

23. The method of claim 21 wherein the patient-chosen parameter includes an input from the patient characterizing the patient's condition.

24. The method of claim 23 wherein the patient input is a rating of at least one of: pain experienced by the patient; involuntary movement; and desire for a drug bolus.

25. The method of claim 21 wherein the non-variable parameter of the function includes a percent rate of change of at least one of a quantitative characteristic of drug delivery in a previous time slot.

26. The method of claim 21 wherein the non-variable parameter of the function includes an increment of change of at least one of a quantitative characteristic of drug delivery in a previous time slot.

27. The method of claim 21 wherein the time slots are of unequal duration.
